(19) United States
(12) Patent Application Publication
(10) Pub. No.: US 2010/0274144 A1
(43) Pub. Date: Oct. 28, 2010

(54) MEASUREMENT CIRCUIT FOR HEART RATE VARIABILITY

(75) Inventors: WEI-CHIH HU, CHUNG LI (TW); CHAO-FENG CHANG, CHUNG LI 32023 (TW)

Correspondence Address: ROSENBERG, KLEIN & LEE, 3458 ELLICOTT CENTER DRIVE-SUITE 101, ELLICOTT CITY, MD, 21043, US

(73) Assignee: CHUNG YUAN CHRISTIAN UNIVERSITY, CHUNG LI (TW)

(21) Appl. No.: 12/726,587

(22) Filed: Mar. 18, 2010

(30) Foreign Application Priority Data
Apr. 28, 2009 (TW) 098114100

(51) Int. Cl. A61B 5/024; A61B 5/04

(52) U.S. Cl. 600/500; 600/515

(57) ABSTRACT

The present invention relates to a measurement circuit for heart rate variability, which uses a measurement circuit for photoplethysmographic (PPG) signal to measure an ear and produces a first measured signal, a measurement circuit for electrocardiographic (ECG) signal to measure a second measured signal, an audio processing unit to produces a sound signal, a control and processing unit for controlling the audio processing unit to play the sound signal, for receiving the first measured signal to produce a corresponding first waveform diagram, and for receiving the second measured signal to produce a corresponding second waveform diagram. Thereby, nervousness and impatience of a person under test can be eliminated, and hence the real heart rate variability of the person under test can be measured.

### FIELD OF THE INVENTION

The present invention relates to a measurement circuit, and particularly to a measurement circuit for heart rate variability.

### BACKGROUND OF THE INVENTION

Heart rate is the frequency at which the heart beats, and its unit is beats per minute (BPM). In 1981, Akselrod published a method for giving the characteristic power spectrum of heart rate variability (HRV) by fast Fourier transform, where the heart rate variability is the difference between each heartbeat interval, namely, the variations in heart rates or heartbeat intervals. The characteristic power spectrum of HRV corresponds to the physiological mechanisms of autonomic nervous systems. Long-term HRV can represent if a person has Dysautonomia or not as well as the health condition of the heart functions.

The autonomic nervous system is a part of the peripheral nervous system, and controls the functions of organs. The autonomic nervous system is divided into two types: the sympathetic nervous system and the parasympathetic nervous system. The sympathetic nervous system is dominant when pressure exists, and prepares the body while facing pressure and consumes energy. On the contrary, the parasympathetic nervous system is dominant while resting and convalescing, and accelerates and regulates processes such as digestion and growth. In order to let the body rest and convalesce, its activities can preserve energy.

Currently, the most effective method for evaluating the activities of the autonomic nervous system is through the analysis of HRV. The characteristics of HRV vary according to the interaction and regulation of the sympathetic and parasympathetic nervous systems. Thereby, medically, HRV is used to study the regulation of autonomic nervous systems. That is to say, HRV can be used to judge if a person's autonomic nerves are disordered. In addition, HRV can also represent the health of the heart: low HRV means high risk in heart disease. Accordingly, the HRV characteristics can be used to judge or treat multiple diseases, such as arrhythmia, diabetes, and melancholia.

The arrhythmia means abnormal heartbeats, including variations in heartbeat intervals and too fast or too slow heart rates. In addition to arrhythmia due to heart diseases, changes in breathing cause arrhythmia as well. For example, when a person inhales deeply, his heart rate will increase; when he exhales, his heart rate will decrease. These are normal physiological phenomena. Besides, when one exercises, his heart rate increases; when he rests or sleeps, the heart rate decreases. Furthermore, heart rate and its rhythm also change owing to excitement of autonomic nervous system, stimulation by coffee or tea, fever, nervousness, pressure, pain, anoxia, medicine.

When arrhythmia occurs, the symptoms can be none or slight such as feeling acceleration of heartbeats or irregular heartbeats. The symptoms can also be as severe as shock, faint, or even sudden death. Many sudden death patients exhibit no symptoms. Sudden death can even happen to young people. It is regarded in the medical field that in addition to analysis of past cases, sudden lowering of HRV can be used as a predictive indication of diseases. Especially, for busy people, by monitoring of long-term HRV, if the HRV is too low or is lowering gradually, they should take rest immediately for reducing the possibility of sudden death.

HRV can be an indication of the treatment effect for diabetes. In the early phase of diabetes, though the blood sugar is maintained in the normal range, the HRV is lowering gradually. In the middle and last phases of diabetes, the patients can possibly have diabetic neuropathy at the same time. Then the sympathetic and parasympathetic fine fibers start necrotizing. The patients will exhibit dysautonomia symptoms of vertigo (low blood pressure), palpitations, night sweat, and diarrhea. By long-term HRV measurement, it is found that the HRV deviates from original baseline. The treatment effects can be evaluated by the measurement as well.

Moreover, HRV can be used to judge morbidity of melancholia. Melancholia is a medical disease, not just depression only. Tens of millions of people suffer from this disease. Females have twice the possibility of having melancholia than males. Patients of heart disease, paralysis, cancer, and diabetes have higher probability o having melancholia. The HRV of these usually patients exhibits active and low values. According to scientific literature, many prescription drugs of western medicine can improve symptoms of melancholia. According to estimation, 80% to 90% of melancholia patients can be totally cured by professional pharmaceutical therapy and psychotherapy. If long-term HRV is used to trace curative effect, melancholia can be fully healed.

To acquire HRV information, it is not necessary to analyze the details of an electrocardiogram. If the period of heartbeats is given, HRV information can be deduced accordingly. It takes a period of time, around 10 minutes, to measure HRV. It is not possible to know the result in a short time. First, the heartbeat period is given by the electrocardiographic signals. After re-sampling, perform fast Fourier transform to the sampled data for giving the power spectrum of heart rate variability. According to the power spectrum of heart rate variability, the high-frequency (0.15-0.4 Hz) power and low-frequency (0.04-0.15 Hz) power are given. The variation in high- and low-frequency power can be used as the indication of activity of autonomic nerves.

However, in the long-term measurement, if the person under test concentrates in the measurement itself, he might feel nervous or impatient, and thus natural physiological information cannot be given. It is easier to observe problems in heart by long-term measurement. If the measurement is performed during a short term, considering the nervousness or impatience of the person under test, some diseases, such as occasional arrhythmia, cannot be observed.

Accordingly, the present invention provides a measurement circuit for heart rate variability, which can make the person under test less nervous or less impatient while measuring. Thereby, the real heart rate variability of the person under test can be measured and giving natural heart rate and heart rate variability but not heart rate variability under nervous conditions.

### SUMMARY

An objective of the present invention is to provide a measurement circuit for heart rate variability, which uses a measurement circuit for photoplethysmographic (PPG) signal and a measurement circuit for electrocardiographic (ECG) signal to measure various physiological signals of a human body simultaneously, and thus improving convenience of measuring physiological signals.

Another objective of the present invention is to provide a measurement circuit for heart rate variability, in which a measurement circuit for photoplethysmographic signal is set in an earpiece. When the measurement circuit for photoplethysmographic signal and a measurement circuit for electrocardiographic signal measure the physiological signals of a person under test, the earpiece can play sound signals for detracting the person under test from the measurement circuit for photoplethysmographic signal and the measurement circuit for electrocardiographic signal, and thus eliminating nervousness and impatience of the person under test. Thereby, the real heart rate variability of the person under test can be measured.

Still another objective of the present invention is to provide a measurement circuit for heart rate variability, which integrates an audio processing unit and a control and processing unit into a chip for shrinking the volume of the measurement circuit for heart rate variability, and hence reducing the manufacturing costs.

The measurement circuit for heart rate variability according to the present invention comprises a measurement circuit for photoplethysmographic signal, a measurement circuit for electrocardiographic signal, an audio processing unit, and a control and processing unit. The measurement circuit for photoplethysmographic signal measures an ear of a person under test and produces a first measured signal. The measurement circuit for electrocardiographic signal measures the physiological status of the body of the person under test and produces a second measured signal. The audio processing unit produces a sound signal and transmits the sound signal to the ear of the person under test. The control and processing unit controls the audio processing unit to play the sound signal, and receives the first measured signal for producing a corresponding first waveform diagram. Besides, the control and processing unit also receives the second measured signal for producing a corresponding second waveform diagram. Thereby, the audio processing unit plays the sound signal for detracting the person under test from the measurement circuit for photoplethysmographic signal and the measurement circuit for electrocardiographic signal, and thus eliminating nervousness and impatience of the person under test. Accordingly, the real heart rate variability of the person under test can be measured.

### BRIEF DESCRIPTION OF THE DRAWINGS

FIG. 1 shows a block diagram according to a preferred embodiment of the present invention;

FIG. 2 shows a block diagram of a processing unit for photoplethysmographic (PPG) signal according to a preferred embodiment of the present invention;

FIG. 3 shows a block diagram of a processing unit for electrocardiographic (ECG) signal according to a preferred embodiment of the present invention;

FIG. 4 shows a flowchart of analyzing the measured signals according to a preferred embodiment of the present invention;

FIG. 5 shows a structural schematic diagram of an earpiece for measuring heart rate variability according to a preferred embodiment of the present invention;

FIG. 6 shows a schematic diagram of an earpiece set in an ear according to a preferred embodiment of the present invention; and

FIG. 7 shows a schematic diagram of performing photoplethysmography according to a preferred embodiment of the present invention.

### DETAILED DESCRIPTION

In order to make the structure and characteristics as well as the effectiveness of the present invention to be further understood and recognized, the detailed description of the present invention is provided as follows along with preferred embodiments and accompanying figures.

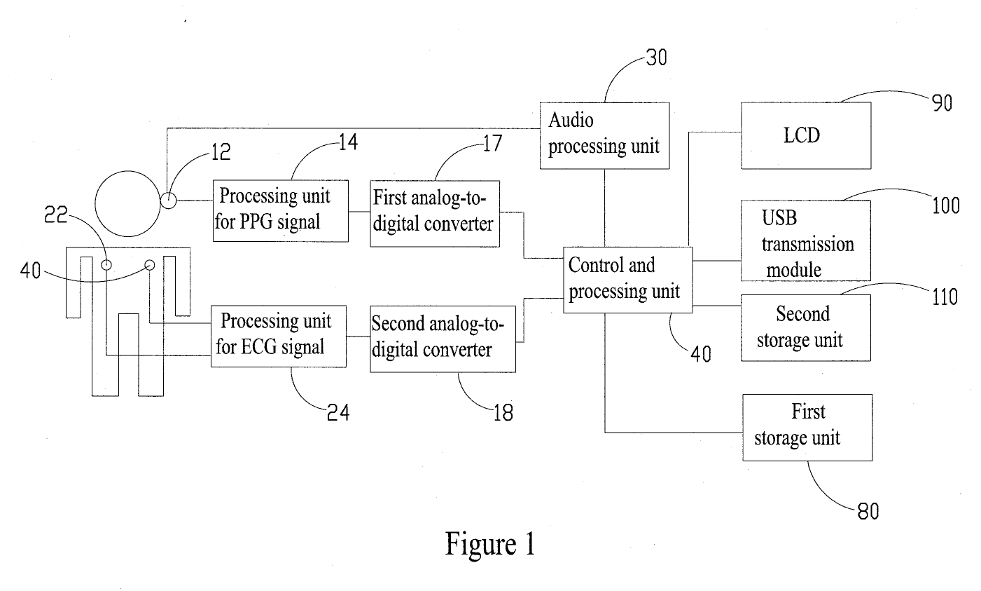

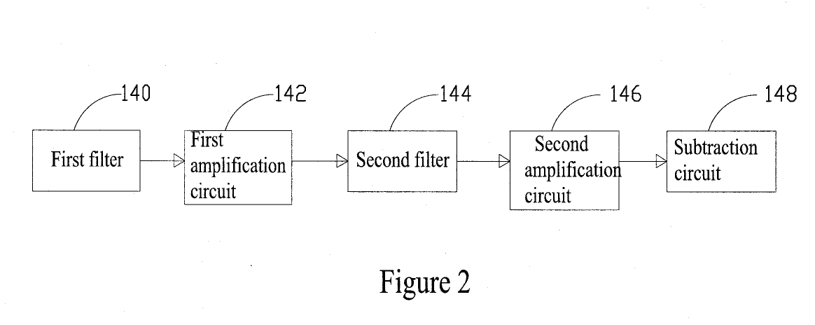

FIG. 1 shows a block diagram according to a preferred embodiment of the present invention. As shown in the figure, the measurement circuit for heart rate variability (HRV) according to the present invention comprises a measurement circuit for photoplethysmographic (PPG) signal 10, a measurement circuit for electrocardiographic (ECG) signal 20, an audio processing unit 30, and a control and processing unit 40. The measurement circuit for PPG signal 10 measures an ear 70 of a person under test (referring to FIG. 2) and produces a first measured signal. The measurement circuit for ECG signal 20 measures the physiological status of the body of the person under test and produces a second measured signal. The audio processing unit 30 produces a sound signal and transmits the sound signal to the ear 70 of the person under test. The control and processing unit 40 controls the audio processing unit 30 to play the sound signal, and receives the first measured signal for producing a corresponding first waveform diagram. Besides, the control and processing unit 40 also receives the second measured signal for producing a corresponding second waveform diagram. Thereby, the audio processing unit 30 can produce the sound signal and transmits the sound signal to the ear 70 of the person under test via the transmission path of the measurement circuit for PPG signal 10. Hence, the person under test can be relaxed, eliminating nervousness and impatience while testing. Accordingly, the real HRV of the person under test can be measured.

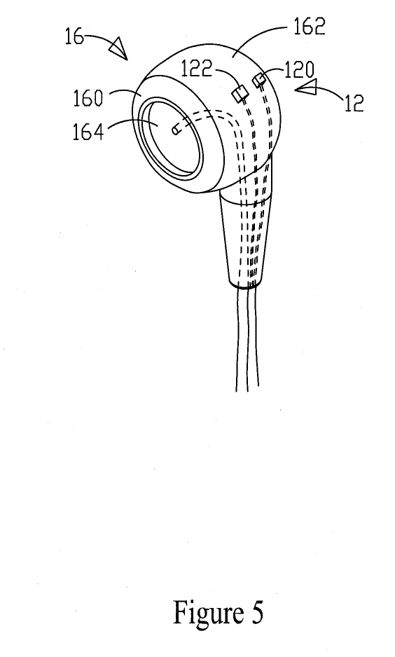

The measurement circuit for PPG signal 10 includes a measurement unit for PPG signal 12 and a processing unit for PPG signal 14. The measurement unit for PPG signal measures the physiological status of the person under test at his ear 70 and produces a first physiological signal. The processing unit 14 for PPG signal 14 receives and processes the first physiological signal, and produces a first measured signal. According to a preferred embodiment of the present invention, the measurement unit for PPG signal 12 can be set in an earpiece 16 (as shown in FIG. 5). The earpiece 16 is placed into the ear 70 of the person under test, and accommodates the measurement unit for PPG signal 12 and a speaker 164 (as shown in FIG. 5). The measurement unit for PPG signal 12 of the measurement circuit for PPG signal 10 includes a light source 120 and a photodetector 122. The light source 120 is set on one side of the earpiece 16, and illuminates the skin of the ear 70 and produces reflection light. The photodetector 122 is in the earpiece 16 as well and is on the same side of the light source 120. The photodetector 122 receives the reflection light, and transmits the reflection light to the processing unit for PPG signal 14. The light source 120 can emit light through an oscillator (not shown in the figure). According to the embodiment of the present invention, a 555 oscillator is used for emitting light.

The audio processing unit 30 produces audio signals and transmits the audio signals to the earpiece 16. Thereby, the person under test can listen to the music while measuring photoplethysmograph, and the real HRV of the person under test can be measured. The processing unit for PPG signal 14 receives the first physiological signal and produces the first measured signal according to the first physiological signal.

FIG. 2 shows a block diagram of a processing unit for photoplethysmographic (PPG) signal according to a preferred embodiment of the present invention. As shown in the figure, the processing unit for PPG signal 14 comprises a first filter 140, a first amplification circuit 142, a second filter 144, a second amplification circuit 146, and a subtraction circuit 148. The first filter 140 filters the reflected light transmitted by the photodetector 122, and produces a first filter signal. The first amplification circuit 142 amplifies the first filter signal. The second filter 144 filters the amplified first filter signal, and produces a second filter signal. The second amplification circuit 146 amplifies the second filter signal. The subtraction circuit 148 adjusts the second filter signal amplified by the second amplification circuit 146, and produces the first measured signal. The first filter 140 is a second-order high-pass Butterworth filter for achieving high-pass filtering effect, which mainly filters out the low-frequency drift component for avoiding low-frequency interferences during measurement. The second filter 144 is a fourth-order low-pass Butterworth filter for achieving low-pass filtering effect and avoiding high-frequency interferences, which are mainly appliance noises at 60 Hz, during measurement. The frequency of PPG signals is under 10 Hz. Thereby, the cutoff frequency is set at 10 Hz for filtering out the 60 Hz signals and the second filter 144 acts as a pre-filter of 60 Hz. Besides, the processing unit for PPG signal 14 can further set a T-notch filter after the second filter 144 for filtering out specific frequency of a unknown signal. In the present system, the specific frequency is aimed at 60 Hz power noises.

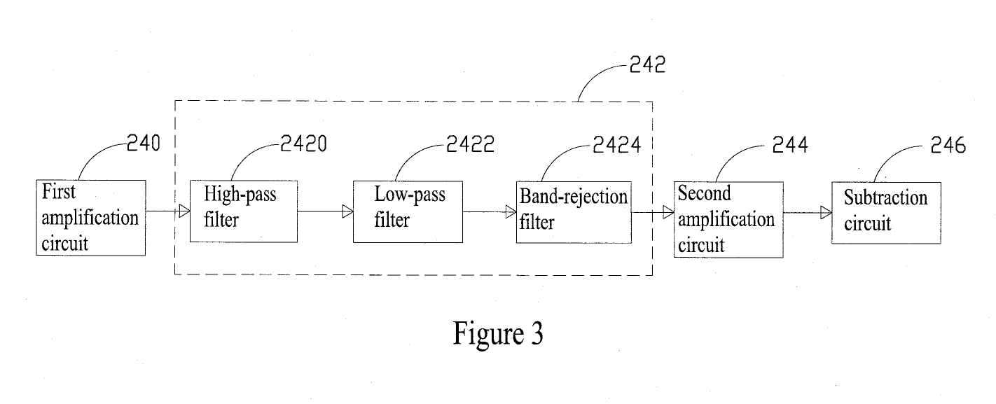

FIG. 3 shows a block diagram of a processing unit for electrocardiographic (ECG) signal according to a preferred embodiment of the present invention. As shown in the figure, the measurement circuit for ECG signal 20 includes a measurement unit for ECG signal 22 and a processing unit for ECG signal 24. The measurement unit for ECG signal 22 measures the physiological status of a human body and produces a second physiological signal. The processing unit for ECG signal 24 receives and processes the second physiological signal and produces the second measured signal. The processing unit for ECG signal 24 includes a first amplification circuit 240, a filter module 242, a second amplification circuit 244, and a subtraction circuit 246. The first amplification circuit 240 amplifies the signals of the physiological status of the human body, and produces a first amplification signal. The filter module 242 filters the first amplification signal and produces a filtered signal. The second amplification circuit 244 amplifies the filtered signal, and produces a second amplification signal. The subtraction circuit 246 adjusts the DC level of the second amplification signal, and produces the second measured signal. The filter module 246 includes a high-pass filter 2420, a low-pass filter 2422, and band-rejection filter 2424. The high-pass filter 2420 filters the low-frequency portion of the first amplification signal of the first amplification circuit 240. The low-pass filter 2422 filters the high-frequency portion of first amplification signal filtered by the high-pass filter 2420. The band-rejection filter 2424 filters the band of the first amplification signal filtered by the low-pass filter 2422 and produces the filtered signal.

In addition, the measurement circuit for heart rate variability according to the present invention further comprises a first analog-to-digital converter 17 and a second analog-to-digital converter 18. The first analog-to-digital converter 17 converts an analog signal of the first measured signal to a digital signal of the first measured signal, and transmits the digital signal to the control and processing unit 40. Likewise, The second analog-to-digital converter 18 converts an analog signal of the second measured signal to a digital signal of the second measured signal, and transmits the digital signal to the control and processing unit 40.

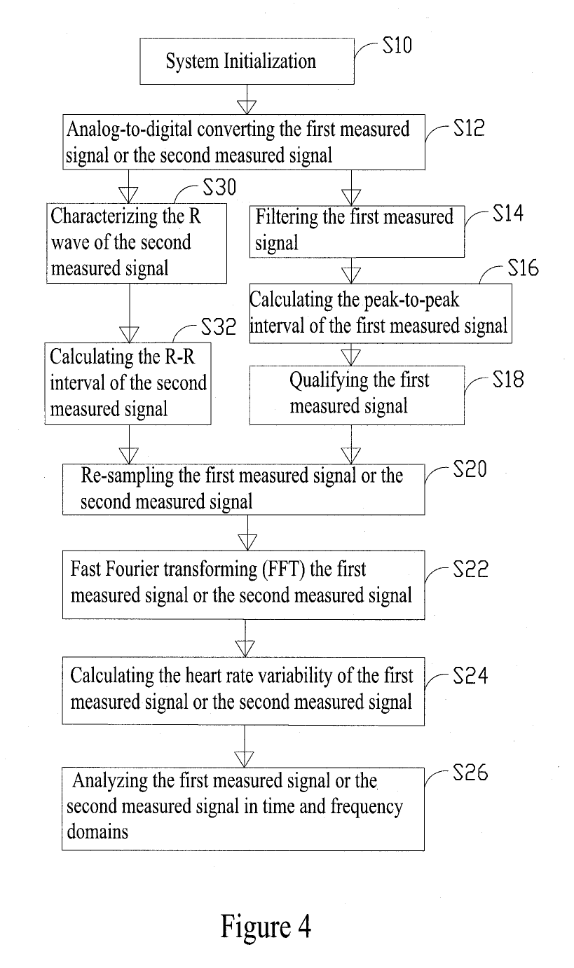

Referring back to FIG. 1, the control and processing unit 40 receives and analyzes the first and second measured signals measured by the measurement circuit for PPG signal 10 and the measurement circuit for ECG signal 20, respectively, and hence gives the HRVs of the first and second measured signals. In the following, the method how the control and processing unit 40 analyzes the first and second measured signals will be described. FIG. 4 shows a flowchart of analyzing the measured signals according to a preferred embodiment of the present invention. As shown in the figure, the step S10 is executed for performing system initialization. Then, the step S12 is executed for analog-to-digital converting the first measured signal or the second measured signal. If the control and processing unit 40 received the first measured signal first, the step S14 is executed for filtering digitally the first measured signal. In this step, because the PPG signal in the ear is small and is prone to being influenced by noise on the analog side, a low-pass 10 Hz digital filter is added on the digital side for filtering out excess noise. Thereby, the peaks of the PPG signal can be located with accuracy. Next, the step S16 is executed for calculating the peak-to-peak interval of the first measured signal. Afterwards, the step S18 is executed for qualifying the first measured signal. The most challenging problem while measuring PPG signal in the ear is vibration. The person under test cannot hold still without any movement while being measured. Such tiny movements (vibrations) add some noises into the PPG signal and thereby cause invalid detection of peak-to-peak intervals of the first measured signal. For avoiding such a situation, sifting is performed to the peak-to-peak intervals. Namely, the heart rate of a normal person is between 60 to 100 beats per minutes. By taking the sampling rate of 200 Hz, the number of samples for each peak-to-peak interval should be between 120 and 200 points. Those peak-to-peak intervals with number of samples beyond said range will be abandoned. Besides, if the difference between the newly acquired peak-to-peak interval and the previously acquired one is too large, the newly acquired one will be abandoned too and next one will be searched.

Then, the step S20 is executed for re-sampling the qualified first measured signal. In this step, after the peak-to-peak intervals are extracted, the sequence composed of the peak-to-peak intervals is the heart rate variability signal. Because this signal is non-equal-interval sampled, according to the present invention, the window interpolation method proposed by Berger et. al. in 1986 is adopted for converting the signals to equal-interval sampled HRV signals, and thus facilitating power spectrum analysis. Next, the step S22 is executed for fast Fourier transforming (FFT) the first measured signal and gives the spectrum signal of the HRV signal. After that, the step S24 is executed for calculating the HRV of the first measured signal. Finally, the step S26 is executed for analyzing the first measured signal in time and frequency domains. In this step, a long-term observation is performed on the HRV signal of the first measured signal.

When the control and processing unit 40 receives the second measured signal, after the step S12, the step S30 will be executed for characterizing the R wave of the second measured signal. In this step, the process of automatically detecting R wave includes differentiating and taking the absolute value of the extracted second measured signal, namely, the ECG signal, window averaging, and R wave detection. This is a technique known by the person having ordinary skill in the art, and thereby will not be described in further details. Afterwards, the step S32 is executed for calculating the R-R interval of the second measured signal. Finally, the steps S20 to S26 are executed as described above.

Referring back to FIG. 1, the present invention further comprises a first storage unit 80, which is coupled to the control and processing unit 40. The control and processing unit 40 stores the first and the second measured signals to the first storage unit 80. The first storage unit 80 is a Compact Flash (CF) card. CF cards have the advantages of high storage capacity, small size, high performance, and convenient portability. In addition, they have fast access time and are compatible with multiple computer operating systems. Thereby, CF cards are widely adopted for data recording in data collection and for accessing data between PCs. A CF card includes a controller, a flash memory array, and an access buffer. The embedded intelligent controller greatly simplifies the design of peripheral circuitry. Besides, it also complies with the interface regulations required by PCMCIA (Personal Computer Memory Card International Association) and ATA (Advanced Technology Attachment). The structure of the buffer in a CF card enables the controller therein to access flash memories while communicating with external equipments. This feature increases reliability of data access in a CF card as well as increasing data transmission rate. CF cards support multiple interface modes, including the Memory Mapped mode and the I/O Card mode of PCMCIA, and the True IDE mode of ATA. When powering on, if the OE pin is low, the CF card enters True IDE mode. Then the OE pin is called ATA SEL. On the contrary, if the OE pin is high when powering on, the CF card enters the PCMCIA mode, namely, the Memory Mapped mode or the I/O Card mode. The corresponding modes can be entered by modifying the configuration register.

In addition, the present invention further comprises a liquid-crystal display (LCD) 90, which is coupled to the control and processing unit 40. The control and processing unit 40 transmits the first waveform diagram and the second waveform diagram to the LCD 90 for displaying. The LCD 90 according to the present invention adopts a thin-film transistor liquid-crystal display (TFT-LCD). The TFT-LCD panel can be regarded as a layer of liquid crystal sandwiched between two glass substrates. The top glass substrate is bonded with a color filter, while the bottom glass substrate has transistors thereon. When current passes through the transistors and produces changes in electric field, the liquid-crystal molecules rotates and thereby changes polarity of light. Then the polarizer is used for determining brightness if a pixel. In addition, because bonding between the top glass substrate and the color filter, each pixel has three colors including red, blue, and green, respectively. These pixels emitting red, blue, and green lights form the image of the panel.

The present invention further comprises one or more second storage units 100, which stores multimedia data such as MP3 data. The audio processing unit 30 reads the multimedia data, converts it, and transmits voice signals. Moreover, the present invention further comprises a USB transmission module 110. The audio processing unit 30 transmits the first and second measured signals to the USB transmission module 110 for sending to a computer. The given timing and pulse data and the analyzed heart rate variability data can be displayed by using Borland C++ Builder for editing user interface windows. Besides, the USB transmission module 110 can transmits data between the computer and the first storage unit 80 as well.

The audio processing unit 30 is mainly used for MP3 encoding/decoding and compression/decompression of other audio formats (such as WMA) for digital media players. After the music is played, the audio data stored in the second storage unit 100 will be played for every 130 ms. In addition, while processing the first and second measured signal, the music will not be interrupted.

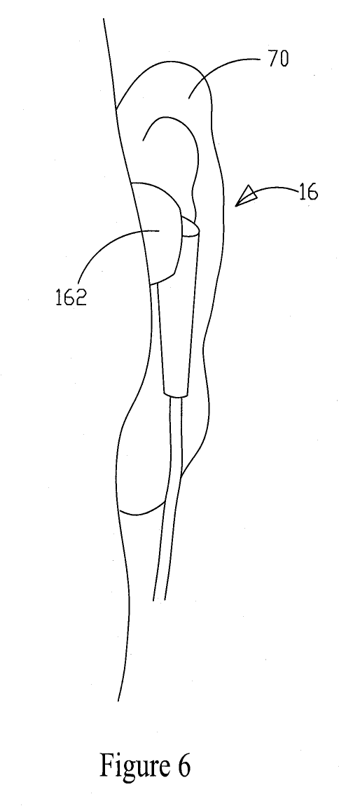

FIG. 5 shows a structural schematic diagram of an earpiece for measuring heart rate variability according to a preferred embodiment of the present invention. In addition, FIG. 6 shows a schematic diagram of an earpiece set in an ear according to a preferred embodiment of the present invention. As shown in the figures, the light source 120 of the measurement unit for PPG signal 12 according to the present invention is set on one side of the earpiece 16. The light source 120 illuminates the skin of the ear 70 and produces reflection light. The photodetector 122 is set on the earpiece 16 and is located on the same side of the light source 120. The photodetector 122 receives the reflection light, and produces the first physiological signal according to the reflection light, and transmits the first physiological signal to the processing unit for PPG signal 14.

The earpiece 16 according to the present invention includes an embedded part 160 and a holding part 162. The embedded part 160 is placed into the ear 70. The earpiece 16 has a speaker 164, which is set in the embedded part 160. The holding part 162 is set on one side of the embedded part 160. The light source 120 and the photodetector 122 are set in the holding part 120. When the earpiece 16 plays music for the person under test, the light source 120 and the photodetector 122 in the earpiece 16 are used for measuring the HRV of the person under test. Thereby, attention of the person under test can be detracted from the HRV measurement circuits, and thus eliminating nervousness and impatience of the person under test. Hence, the real heart rate variability of the person under test can be measured.

The present invention adopts photoplethysmography (PPG) to extract the first physiological signal. According to the method, a light source 120 with a red LED is needed and a photodetector 122 of light-receiving transistor is used as the probe of PPG. The light source 120 includes red light, and can be a red LED with wavelength 640 nm. The photodetector 122 includes a light-receiving transistor. Because the volume of the light-receiving transistor is relatively small, the photodetector 122 and the light source 120 are set in the earpiece 16. It is uneasy for the person under test to aware the location of the probe of PPG. Thereby, when the person under test is testing and listening to the music, his nervousness can be eliminated. Accordingly, the log-term HRV data of the person under test can be measured with better measurement accuracy.

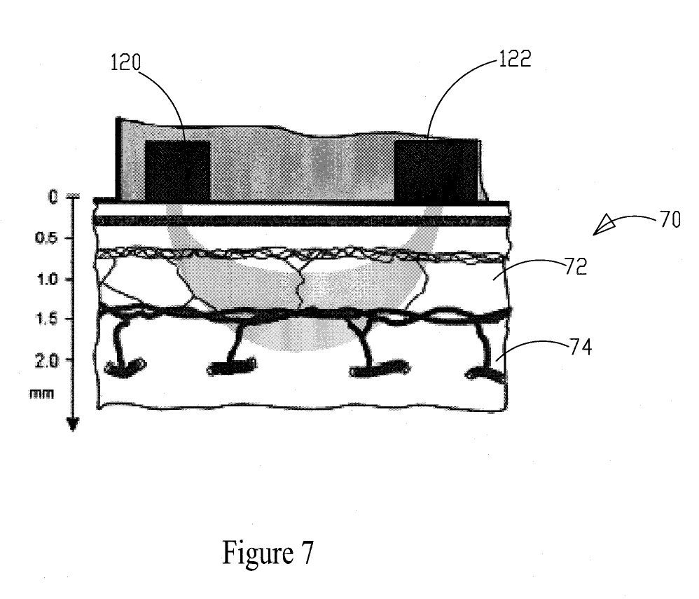

FIG. 7 shows a schematic diagram of performing photoplethysmography (PPG) according to a preferred embodiment of the present invention. As shown in the figure, PPG measures light characteristics by emitting near infrared light source 120 into a selected skin area. When light propagates in the tissue of an organism, it will be absorbed by various absorbing materials such as skin, bones, blood in the artery and the vein. Besides, the artery vessels contain more blood in the systolic period than in the diastolic period. The radii of artery vessels increase as the blood pressure increases, which increase happens only to arteries and arterioles but not to veins. During the systolic period, the absorbability of light increases due an increase of light absorbing materials (such as hemoglobin) as well as an increase of distance traveled by light in the arteries. For the overall absorbability, it acts like an alternating current (AC) component. The AC component helps to identify invariant quantity in the vein blood and in the artery blood, and to identify the difference between light absorbability without pulse component (DC component) such as skin and light absorbability with a pulse component (AC component). The AC component will not exceed 1%˜2% of the DC component. Thereby, PPG refers to receiving waveforms of light signals changing according to time and organism variations. According to the present invention, the light source 120 is emitted to the epidermis 72 of the ear 70. The light passing through the epidermis 72 will then be reflected by the derma 74.

To sum up, the present invention relates to a measurement circuit for heart rate variability, which uses a measurement circuit for photoplethysmographic (PPG) signal to measure an ear and produces a first measured signal, a measurement circuit for electrocardiographic (ECG) signal to measure a second measured signal, an audio processing unit to produces a sound signal, a control and processing unit for controlling the audio processing unit to play the sound signal, for receiving the first measured signal to produce a corresponding first waveform diagram, and for receiving the second measured signal to produce a corresponding second waveform diagram. Thereby, nervousness and impatience of a person under test can be eliminated, and hence the real heart rate variability of the person under test can be measured.

Accordingly, the present invention conforms to the legal requirements owing to its novelty, non-obviousness, and utility. However, the foregoing description is only a preferred embodiment of the present invention, not used to limit the scope and range of the present invention. Those equivalent changes or modifications made according to the shape, structure, feature, or spirit described in the claims of the present invention are included in the appended claims of the present invention.

1. A measurement circuit for heart rate variability, comprising:
a measurement circuit for photoplethysmographic (PPG) signal, measuring an ear, and producing a first measured signal;
a measurement circuit for electrocardiographic (ECG) signal, measuring the physiological status of a person under test, and producing a second measured signal;
an audio processing unit, producing a sound signal, and transmitting the sound signal to the ear; and
a control and processing unit, controlling the audio processing unit, playing the sound signal, receiving the first measured signal and producing a corresponding first waveform diagram, and receiving the second measured signal and producing a corresponding second waveform diagram.

2. The measurement circuit for heart rate variability of claim 1, and further comprising an earpiece, placed in the ear, and holding the measurement circuit for PPG signal.

3. The measurement circuit for heart rate variability of claim 2, wherein the earpiece has a speaker for playing the sound signal.

4. The measurement circuit for heart rate variability of claim 3, wherein the measurement circuit for PPG signal comprises:
a measurement unit for PPG signal; and
a processing unit for PPG signal.

5. The measurement circuit for heart rate variability of claim 4, wherein the measurement unit for PPG signal comprises:
a light source, set on one side of the earpiece, and illuminating the skin of the ear and producing reflection light; and
a photodetector, set on the earpiece and on the same side of the light source, receiving the reflection light, and transmitting the reflection light to the processing unit for PPG signal.

6. The measurement circuit for heart rate variability of claim 4, wherein the earpiece comprises:
an embedded part, placed into the ear, and holding the speaker; and
a holding part, set on one side of the embedded part, and holding the light source and the photodetector.

7. The measurement circuit for heart rate variability of claim 5, wherein the light source includes red light.

8. The measurement circuit for heart rate variability of claim 7, wherein the wavelength of the red light is 640 nm.

9. The measurement circuit for heart rate variability of claim 5, wherein the light source is a red-light LED.

10. The measurement circuit for heart rate variability of claim 5, wherein the photodetector includes a light-receiving transistor.

11. The measurement circuit for heart rate variability of claim 5, wherein the light source passes through the epidermis of the ear to the derma, and the derma reflects the light and produces the reflection light.

12. The measurement circuit for heart rate variability of claim 4, wherein the processing unit for PPG signal further comprises:
a first filter, filtering the reflection light, and producing a first filter signal;
a first amplification circuit, amplifying the first filter signal;
a second filter, filtering the amplified first filter signal, and producing a second filter signal;
a second amplification circuit, amplifying the second filter signal; and
a subtraction circuit, adjusting the amplified second filter signal amplified by the second amplification circuit, and producing the first measured signal.

13. The measurement circuit for heart rate variability of claim 1, wherein the measurement circuit for ECG signal comprises:
a first amplification circuit, amplifying the physiological status signal of the person under test, and producing a first amplification signal;
a filter module, filtering the first amplification signal, and producing a filter signal;
a second amplification circuit, amplifying the filter signal, and producing a second amplification signal; and
a subtraction circuit, adjusting the direct-current (DC) level of the second amplification signal, and producing the second measured signal.

14. The measurement circuit for heart rate variability of claim 13, wherein the filter module includes:
a high-pass filter, filtering out the low-frequency signals of the first amplification signal;
a low-pass filter, filtering out the high-frequency signals of the first amplification signal filtered by the high-pass filter;
a band-rejection filter, filtering out a band of the first amplification signal filtered by the low-pass filter, and producing the filter signal.

15. The measurement circuit for heart rate variability of claim 1, and further comprising an analog-to-digital converter, converting an analog signal of the first measured signal to a digital signal of the first measured signal, and transmitting the digital signal to the control and processing unit.

16. The measurement circuit for heart rate variability of claim 1, and further comprising an analog-to-digital converter, converting an analog signal of the second measured signal to a digital signal of the second measured signal, and transmitting the digital signal to the control and processing unit.

17. The measurement circuit for heart rate variability of claim 1, and further comprising a first storage unit, coupled to the control and processing unit, and storing the first measured signal and the second measured signal.

18. The measurement circuit for heart rate variability of claim 17, wherein the first storage unit includes a Compact Flash (CF) card.

19. The measurement circuit for heart rate variability of claim 1, and further comprising one or more second storage units, storing multimedia data, and the audio processing unit reading and converting the multimedia data and playing the sound signal.

20. The measurement circuit for heart rate variability of claim 1, and further comprising a liquid crystal display (LCD), coupled to the control and processing unit, and the control and processing unit transmitting the first waveform diagram and the second waveform diagram to the LCD for displaying.

21. The measurement circuit for heart rate variability of claim 20, wherein the LCD is a thin-film transistor liquid-crystal display (TFT-LCD).

22. The measurement circuit for heart rate variability of claim 1, and further comprising a USB transmission module, the control and processing unit transmitting the first measured signal and the second measured signal to the USB transmission module for further transmitting to a computer.

23. The measurement circuit for heart rate variability of claim 1, wherein the audio processing unit and the control and processing unit are integrated into a chip.
